(19) United States
(12) Patent Application Publication
(10) Pub. No.: US 2006/0121501 A1
(43) Pub. Date: Jun. 8, 2006

(54) Method for immobilizing molecular probes to a semiconductor oxide surface

(75) Inventors: Harry S. Jabs, Sugar Land, TX (US); Dennis Wright, Lovelady, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: Stanislaw R. Burzynski

(21) Appl. No.: 11/251,611

(22) Filed: Oct. 14, 2005

Related U.S. Application Data
(60) Provisional application No. 60/621,585, filed on Oct. 22, 2004.

(51) Int. Cl. C12Q 1/68; G01N 1/31; C12M 1/34

(52) U.S. Cl. 435/6; 435/287.2; 422/82.03; 427/2.13

(57) ABSTRACT

A method immobilizing molecules on the surface of a semiconductor oxide substrate by forming stable bonds with hydrazine bound to the surface is disclosed. Also disclosed is a FET sensor for sensing target molecules in a solution. The FET is modified with molecular probes immobilized on the sensor surface via hydrazone bonds. The immobilized molecular probes are available to bind target molecules present in a solution and the FET will respond to the binding event.

### PRIORITY CLAIM

This application claims priority to U.S. provisional application 60/621,585 filed Oct. 22, 2004 and incorporates by reference herein the entire contents thereof.

### FIELD OF THE INVENTION

The invention relates to analytical technology and biotechnology and, more specifically, to detectors for molecular targets, such as oligonucleotides, antibodies, antigens, proteins, peptides, enzymes, hormones, metabolites, or drug substances. In particular, a detector based on a field effect transistor for detecting DNA hybridization is disclosed.

### DESCRIPTION OF RELATED ART

U.S. Pat. No. 6,159,695, by McGovern et al. discloses methods of immobilizing oligonucleotides and other biomolecules on solid substrates. According to McGovern et al., substrates that have hydroxyl groups on their surfaces can be first silanized with a trichlorosilane containing 2-20 carbon atoms in its hydrocarbon backbone, terminating in a protected thiol group. The oligonucleotides or other biomolecules are first connected to a tether consisting of a hydrocarbon or polyether chain of 2-20 units in length, which terminates in a thiol group. This thiol may be further modified with a halobenzylic-bifunctional water-soluble reagent, which allows the biomolecule conjugate to be immobilized onto the surface thiol group by a permanent thioether bond. Alternatively, the oligonucleotide-tether-thiol group can be converted to a pyridyldisulfide functionality, which attaches to the surface thiol by a chemoselectively reversible disulfide bond. The permanently bound oligonucleotides are immobilized in high density compared to other types of thiol functionalized silane surface and to the avidin-biotin method.

“Silanized nucleic acids: a general platform for DNA immobilization” by Anil Kumar, et al., Nucleic Acids Research, 2000 Vol. 28, discloses a method for simultaneous deposition and covalent cross-linking of oligonucleotide or PCR products on unmodified glass surfaces by conjugating an active silyl moiety onto oligonucleotides. The silanized molecules are then immobilized onto glass.

“Electronic detection of DNA by its intrinsic molecular charge” by Jüirgen Fritz, et al., PNAS 2002, 14142-46, discloses the selective and real-time detection of label-free DNA using a field effect transistor (FET). The DNA is electrostatically immobilized on a polylysine layer, which is itself electrostatically immobilized on the surface of the FET.

U.S. Pat. No. 6,482,639, by Snow et al. discloses a molecular recognition-based electronic sensor, which is a gateless, depletion mode field effect transistor consisting of source and drain diffusions, a depletion-mode implant, and insulating layer chemically modified by immobilized molecular receptors that enables miniaturized label-free molecular detection amenable to high-density array formats. The conductivity of the active channel modulates current flow through the active channel when a voltage is applied between the source and drain diffusions. The conductivity of the active channel is determined by the potential of the sample solution in which the device is immersed and the device-solution interfacial capacitance. The conductivity of the active channel modulates current flow through the active channel when a voltage is applied between the source and drain diffusions. The interfacial capacitance is determined by the extent of occupancy of the immobilized receptor molecules by target molecules. Target molecules can be either charged or uncharged. Change in interfacial capacitance upon target molecule binding results in modulation of an externally supplied current through the channel.

U.S. Pat. No. 6,803,228, by Caillat et al. discloses a method to produce a biochip and to a biochip composed of biological probes grafted onto a conductive polymer. The method comprises: a) structuring of a substrate so as to obtain on the substrate microtroughs comprising in their base a layer of material capable of initiating and promoting the adhesion onto the layer of a film of a pyrrole and functionalized pyrrole copolymer by electropolymerisation, b) collective electropolymerisation, so as to form an electropolymerized film of a pyrrole and functionalized pyrrole copolymer on the base of the microtroughs, c) direct or indirect fixation of functionalized oligonucleotides by microdeposition or a liquid jet printing technique.

### SUMMARY OF THE INVENTION

One aspect of the present invention is a method of attaching a molecule to the surface of a substrate, comprising selecting a substrate having at least one surface comprising a semiconductor oxide; contacting the selected surface with a hydrazine compound to produce exposed hydrazine groups bound to the substrate surface; selecting a molecule capable of forming a stable bond with a primary amine of the exposed hydrazine group; and contacting the hydrazine groups with the selected molecule. As used herein, “hydrazine groups bound to the substrate surface” means one or more —NH_{X}NH₂ groups bound to the surface. Depending on the mode of bonding with the surface, x can be 0 or one. For example x is 0 if the hydrazine forms a double bond with the surface. Examples of suitable substrates include substrates having a surface comprising a semiconductor oxide, or other surfaces with double bonds to oxygen. The selected surface is preferably composed of silicon dioxide or germanium dioxide. Further, the selected surface preferably excludes organic polymeric materials, such polycarboxylates, polyvinyls or polyacetates. The selected molecule attached to the hydrazine group is typically a molecular probe, such as, but not limited to, antibodies, antigens, oligonucleotides, proteins, peptides, enzymes, enzyme substrates, metabolites, hormones, or drug compounds. The selected molecule comprising a functional group such as an aldehyde, ketone, carboxy group or urea group that is capable of reacting with the primary amine of the exposed hydrazine groups to form a hydrazone bond. In this fashion the selected molecular probe is immobilized to the substrate surface.

A further aspect of the invention is a method of attaching an oligonucleotide to silicon dioxide, comprising contacting the silicon dioxide with hydrazine dihydrochloride to produce hydrazine groups on the silicon dioxide; selecting an oligonucleotide having a 5′-aldehyde functional group; and contacting the hydrazine groups with the selected oligonucleotide.

A further aspect of the invention is a method of activating a substrate for the immobilization of molecules, the method comprising selecting a substrate having at least one surface comprising a semiconductor oxide; contacting the selected surface with a hydrazine compound to produce hydrazine groups on the selected surface.

A still further aspect of the invention is a derivatized semiconductor oxide surface for the immobilization of molecules, comprising hydrazine groups bonded to the semiconductor oxide surface.

A still further aspect of the invention is a sensor for detecting target molecules comprising: a field effect transistor (FET) having a source implant and a drain implant that are spatially arranged within a semiconductor structure, said source and drain being separated by an active channel; a dielectric layer covering said active channel, said dielectric layer having a bottom surface in contact with the active channel and a top surface in contact with a sample solution, wherein the top surface is modified with molecular probes immobilized to the top surface via hydrazone bonds, and wherein the immobilized molecular probes being available to bind target molecules present in the sample solution, wherein said FET is imbedded in a substrate with said receptor modified dielectric layer exposed; and a reference electrode in contact with said sample solution, wherein said substrate is biased with respect to said reference electrode.

### DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1 shows a FET sensor according to the present invention.

FIG. 2 shows a FET sensor interfaced with a flow cell.

FIG. 3 shows a FET-flow cell assembly interfaced with electronics for providing constant drain current and constant drain voltage.

FIG. 4 shows the gate bias response when a FET sensor was derivatized with hydrazine groups bound to the surface of the FET.

FIG. 5 shows the gate bias response to DNA hybridization on the surface of a FET sensor.

FIG. 6 shows gate bias changes on a post-hybridization FET sensor.

FIG. 7 shows the gate bias response to DNA hybridization on the surface of a FET sensor.

FIG. 8 shows gate bias changes on a post-hybridization FET sensor.

### DETAILED DESCRIPTION OF THE PREFERRED EMBODIMENTS

One aspect of the present invention is a method of attaching molecules to the surface of a substrate. According to one embodiment, the method comprises selecting a substrate having at least one surface that reacts with a hydrazine compound to yield hydrazine groups bound to the surface. As used herein, “hydrazine groups bound to the surface” means one or more —NHₓNH₂ groups bound to the surface. Depending on the mode of bonding with the surface, x can be 0 or one. For example x is 0 if the hydrazine forms a double bond with the surface. Examples of suitable substrates include substrates having a surface comprising a semiconductor oxide, or other surfaces with double bonds to oxygen, such as silicon dioxide or germanium dioxide. According to one embodiment, the surface does not comprise an organic polymer. According to another embodiment, the surface does not comprise a carboxylate-modified polymer, for example latex. Suitable hydrazine compounds include hydrazine dihydrochloride, hydrazine sulfate, and hydrazine.

The method of attaching molecules to a semiconductor oxide substrate surface comprises contacting the surface with a hydrazine compound to produce hydrazine groups on the surface. According to one embodiment of the invention, the hydrazine compound is provided as an aqueous solution. Preferred examples of such solutions include aqueous hydrazine dihydrochloride solutions having a concentration of about 0.1M to about 2.5M, more preferably about 1M to about 2M, and even more preferably about 2M.

The surface is typically contacted with the hydrazine solution for a period of time of about 10 minutes to about 18 hours. The present invention provides an activated surface, that is, a surface derivatized with active hydrazine groups.

According to one embodiment, the method further comprises selecting a molecule (referred to herein as a probe molecule) to attach to the hydrazine-derivatized surface. Generally any molecule containing a functional group capable of forming a stable chemical bond with a hydrazine group can be attached to a surface using the method of the present invention. Examples of functional groups that form stable chemical bonds with hydrazine groups include aldehydes, ketones, carboxylic acids, ureas, and acyl groups. Examples of types of molecules that can be attached to a substrate surface using the method of the present invention include poly-nucleic acid molecules such as oligonucleotides and polynucleotides; polypeptide molecules such as proteins and oligopeptides; enzymes, cryptans, crown ethers, ureas and urea derivatives, molecules containing acyl groups capable of hydrazone bond formation.

The method further comprises contacting the derivatized surface with a solution of the probe molecule. It is within the ability of one of skill in the art to determine suitable solvents and concentrations for contacting the derivatized surface with probe molecules depending on the particular probe molecule. For example, if the probe molecule is an oligonucleotide, then an example of a suitable solution is an aqueous solution of the oligonucleotide. Examples of preferred embodiments include aqueous solutions of oligonucleotides, wherein the oligonucleotide concentration is about 1 nM to about 80 nM, preferably about 5 nM to about 20 nM, and more preferably about 10 nM. It may also be preferred that such solutions contain a buffer to keep within a particular pH, for example about 4.5 to about 11.5. An example of a suitable buffer is phosphate. Another suitable buffer is tris(hydroxymethyl)aminomethane (tris buffer). Other buffers known in the art are also suitable.

According to one embodiment, the derivatized surface is contacted with the solution of the probe molecule for a period of time of about 1 hr to about 72 hr, preferably about 4 hr to about 48 hr, and more preferably about 8 hr to about 24 hr. For example, the substrate comprising the derivatized surface can be submerged in a solution of the probe molecule. Alternatively, one or more drops of a solution of the probe molecule can be applied to the derivatized surface and allowed to stand in contact with the derivatized surface for a period of time sufficient for the probe molecule to attach to the surface.

One of skill in the art will recognize that one aspect of the present invention is a novel method of attaching molecules to the surface of a semiconductor oxide substrate. One of skill in the art will further appreciate that such a method enables using such surface-modified substrates in analytical and bio-analytical applications. For example, when a probe molecule is chosen that has a specific binding affinity for a relevant target molecule, a substrate that has the probe molecules attached to its surface can be used as a sensor for that target molecule. Such a substrate can be contacted with a medium suspected of containing target molecules and the presence and/or concentration of target molecules can be determined by determining how many, if any, target molecules interact with the surface-bound probe molecules. Any of the optical and electrochemical techniques that are known in the art for probing such solution-surface interactions are contemplated as aspects of the present invention.

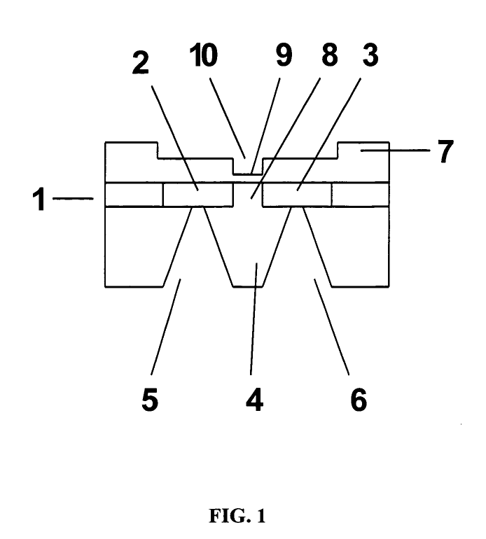

An example of a particular preferred embodiment of a sensor according to the present invention is a detector that uses a field-effect transistor (FET) as a transducer. A schematic diagram of a sensor according to the invention is shown in FIG. 1. The FET comprises an n+ source 2 and an n+ drain 3 embedded within a p+ body 4. Both the source 2 and the drain 3 are equipped with back-side contacts, 5 and 6, respectively, for making electrical contact with the source and drain. A silicon dioxide “gate-oxide” layer 7 covers an n− active channel 8. This gate-oxide layer acts as a dielectric layer. According to a preferred embodiment, probe molecules are immobilized to the surface 9 of the gate oxide layer 7, in accordance with the method described above. The immobilized probe molecules are available to bind target molecules present in the sample solution, which is delivered to the sampling region 10. The observed conductivity of the active channel 8 responds quickly and substantially to changes in the capacitance of the gate oxide layer 7 due to target molecules binding to the probe molecules. One of skill in the art will appreciate that other FET configurations are available and applicable as alternative embodiments of the present invention.

According to one embodiment, the FET is operated in constant drain current and constant drain voltage and the gate bias voltage is used as the transducer signal. In practice, a sample solution containing no, one, or more target molecule species is allowed to contact the sampling region 10. The consequent surface potential represents a gate bias that couples capacitively to the active channel 8, which is itself biased by the source and drain applied potentials. Binding of target molecules (if present) by the immobilized probe molecules changes the capacitive coupling between the channel and the solution, and thus changes channel conductivity. Alternatively, if the target molecule is charged, then a binding event will also change the surface potential at 9, and thereby modulate the charge carrier density in the channel region 8. Such a change will be reflected as a change in gate bias voltage. The gate bias voltage can be measured relevant to a reference electrode present in the solution.

A device according to the present invention can be miniaturized and fabricated by standard microelectronic techniques in high-density arrays for simultaneous detection of multiple target molecules, with sensitivity increasing with miniaturization. Examples of potential uses include, but are not limited to, a genetic assay based in a point of care environment requiring limited instrumentation and performed by non-technically trained personnel to provide important genetic information rapidly and cost-effectively.

According to one embodiment of the invention, discrete samples can be analyzed sequentially. For example, a sample solution possibly containing target molecules can be disposed within sampling region 10 and the gate bias voltage measured to determine if a binding event(s) has occurred. The solution can be delivered via pipette, dropper, or any other means known in the art. The delivery can be by hand or by robotic means. Following the gate bias voltage measurement, the analyte solution can be exchanged for a new analyte solution and a new measurement can be taken.

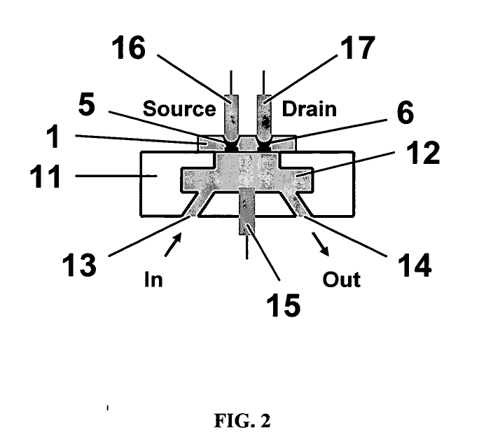

An alternative embodiment for a FET sensor according to the present invention is depicted in FIG. 2. According to this embodiment, the FET sensor is integrated with a flow cell 11 that allows analyte solution to be continuously supplied to the FET senor. The flow cell comprises a FET sensor 1 positioned so that sampling region 10 (not specifically shown) contacts solution contained in cell cavity 12. Contact between FET 1 and flow cell 11 can be maintained by any mechanical means such as clip(s). According to one embodiment, the FET 1 can be sealed to the flow cell 11 using an adhering compound such as silicone or Apiezon TM grease. Flow cell 11 comprises an inlet 13 and an outlet 14 for moving analyte solution to and from cavity 12. Analyte solution can be moved to and from the cell, for example, through tubing connected to 13 and 14 using a syringe or a pump, e.g., a peristaltic pump. According to a preferred embodiment, the fluid flow rate can be varied. According to one embodiment, the fluid flow rate is about 0.05 ml/minute to about 2 ml/minute, more preferably about 0.1 ml/minute to about 1 ml/minute, and even more preferably about 0.3 ml/minute to about 0.7 ml/minute, for example, about 0.5 ml/minute.

This embodiment depicted in FIG. 2 also comprises a reference electrode 15 positioned so that it can contact solution contained in cell cavity 12. According to one embodiment, reference electrode 15 can be used to measure the gate bias. According to one embodiment, reference electrode 15 is a platinum electrode. Alternatively, any reference electrode known in the art, e.g., Ag/AgCl or SCE, can be used. Contact is made with the source and drain via leads 16 and 17, respectively, which are connected to backside contacts 5 and 6.

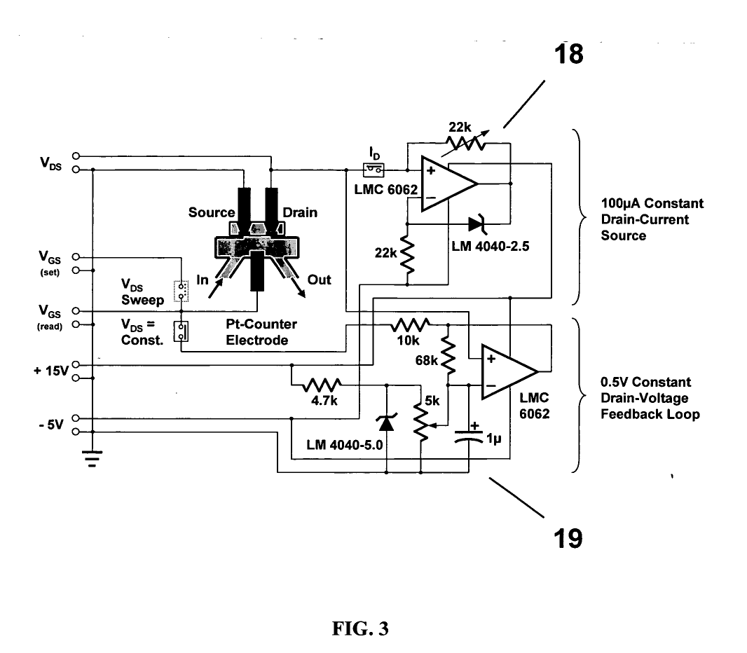

According to one embodiment, the FET 1 is operated with constant drain current and constant drain voltage. FIG. 3 shows an example of electronics for supplying constant drain current and constant drain voltage to FET 1. The embodiment depicted in FIG. 3 has one driver 18 for supplying constant drain current and another driver 19 for maintaining constant drain voltage. The particular values given in FIG. 3 supply a 100 μA constant drain current and a 0.5 V constant drain voltage. It is within the ability of one of skill in the art to design drivers to supply other particular drain current and drain voltage.

A typical experiment using the embodiment of the invention depicted in FIGS. 2 and 3 comprises providing a solution of a hydrazine derivatizing agent to cell cavity 12 vial inlet 13. The derivatizing solution can be maintained in cavity 12 and allowed to contact FET 1 for a period of time sufficient to derivative the surface of FET 1 with active hydrazine groups. Typically, the derivatization of the FET surface will result in a change in the bias potential of the surface and will be reflected in a measurement of the gate bias potential relative to reference electrode 15. The derivatizing solution can then be replaced with a solution of a probe molecule, which is allowed to contact the derivatized surface for a length of time sufficient for the probe molecule to form chemical bonds with the surface-bound active hydrazine groups. Once the probe molecules are bound to the surface, the solution in cavity 12 can be replaced with an analyte solution that possibly contains no, one, or more target molecule species. Binding events can be monitored as a function of observed gate bias potential.

One of skill in the art will appreciate that the embodiment depicted in FIGS. 2 and 3 provide a means of conducting a multitude of binding studies. For example, after the analyte solution has been allowed to contact the surface-bound probe molecules, the analyte solution can be replaced with a competitive binding solution and the kinetics of the competitive binding behavior can be ascertained from changes in the observed gate bias potential.

Likewise, competition for the binding of a target between the surface bound probe molecule and a solution phase competing reaction can be studied. A particularly preferred embodiment is a method of genetic screening, wherein the surface bound probe is a particular nucleotide and the analyte solution possibly contains the complementary sequence for the probe molecule.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Derivatization of a FET Surface with Hydrazine Groups

An FET sensor as depicted in FIG. 1 was integrated in a flow cell apparatus as depicted in FIG. 2, which was integrated with a measuring apparatus as depicted in FIG. 3. The FET was similar to the FET described in “Technology and Measurement of Backside Contacted ISFETs” by B. Jaroszewicz, et al. in Proc. Of the 9^{th} International Conference of Mixed Design, MIXDES 2002, Wroclaw, Poland, June 2002, the entire contents of which are hereby incorporated by reference. The FET had an n-type channel and worked in depletion mode. The FET was operated in constant drain current and constant drain voltage for maximum sensitivity and stability. An electronic circuit according to FIG. 3 provided a driver for maintaining a 100 μA constant drain current and a driver for maintaining a 0.5 V constant drain voltage by controlling the gate potential as needed to maintain these values. Both drivers used operational amplifiers in combination with precision voltage references. Current and voltage were operator adjustable. A jumper option provided switching from computer-controlled gate voltage during characterization sweeps to constant voltage mode during device operation. The circuit board was mounted in close proximity to the device, all of which was copper RF-shielded. The circuit connected through low triboelectric noise RG-174/U coaxial cable to a SC-2345 module breakout box (National Instruments). The box was connected to an IDE I/O card inside a personal computer through a 1 meter long 68-pin shielded cable. A graphical user interface and data acquisition software were programmed in MICROSOFT Visual Basic using National Instrument's Measurement Studio software package.

The flow cell was built from polypropylene round stock. The flow cell had a dead volume of 3.3 μl and was equipped with a platinum reference electrode situated so as to make contact with solution within the cell. The FET was greased with inert vacuum grease (Apiezon M, Apiezon Products, M&I Materials Ltd., Manchester, U.K.) around the outer margin of the front side of the chip where it butted against the cell. Pushpins placed against the backside contacts 5 and 6 pressed the chip against the cell body and facilitated electrical contact. Polypropylene-based PHARMED tubing (0.0449″ ID) was used as intake and discharge tubing. A peristaltic pump was used to move fluids to and from the cell at a flow rate of 0.5 ml/minute.

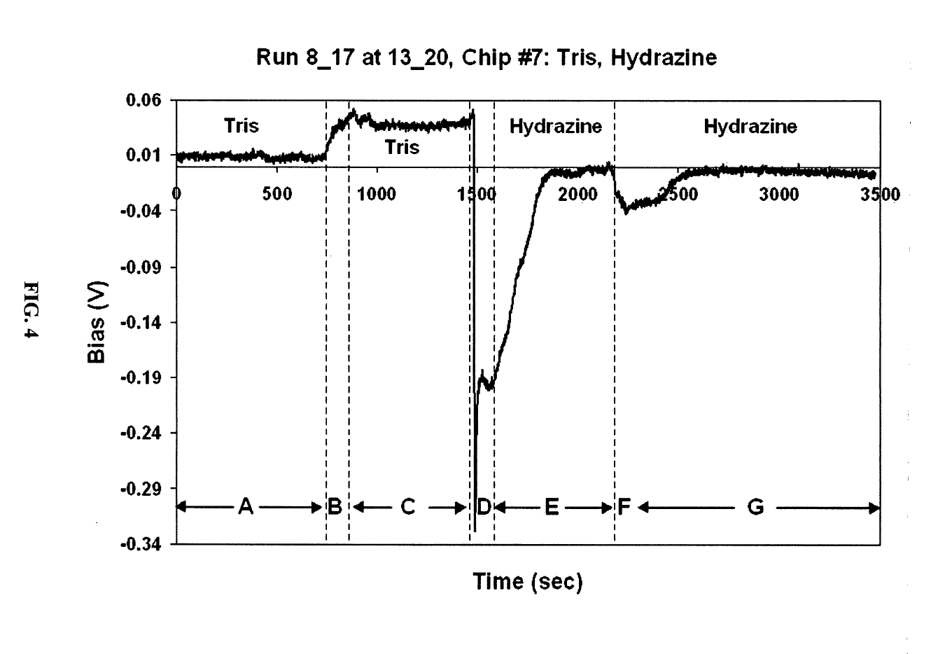

FIG. 4 shows the gate potential response when the FET surface is derivatized with hydrazine molecules. During time interval A, the underivatized FET was in contact with a dead volume of tris buffer (pH 7.1, concentration 100 mM). Fresh tris buffer was pumped during interval B and allowed to contact the FET during time interval C. During time interval D, an aqueous hydrazine dihydrochloride solution (2M) was pumped into the cell. The gate potential response shows a sharp spike, probably due to an air bubble, but then remains at about −0.19 V. There was no pumping during time interval E, and the FET remained in contact with the hydrazine dihydrochloride solution. The voltage potential gradually returned to baseline as the surface derivatization reaction occurred. Pumping of additional hydrazine dihydrochloride was resumed during time interval F, and stopped during time interval G. There was an additional potential response during exposure to the fresh hydrazine dihydrochloride solution, but the magnitude was much less than that of the original exposure. This indicates that the surface of the FET was nearly saturated with hydrazine during the initial exposure to hydrazine dihydrochloride.

### Example 2

### Determination of Nucleic Acid Hybridization with a Field Effect Transistor

A hydrazine-derivatized FET prepared as described in Example 1 was integrated in the flow cell and exposed to a dead volume of a 10 nM solution of a 20-mer of poly-thymidine (poly-T) with a 5′-aldehyde group (SoluLink Biosciences, San Diego, Calif. 92121). The hydrazine-derivatized FET was contacted with the poly-T for 65 hours to produce a hydrazone bond between the surface bound hydrazine and the aldehyde group of the poly-T.

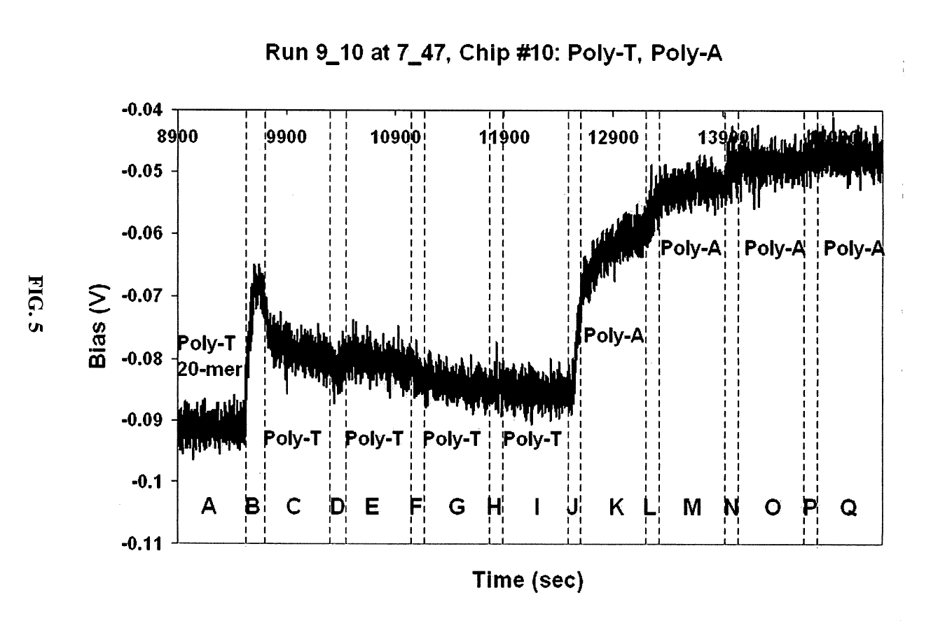

FIG. 5 shows hybridization studies using the poly-T derivatized FET. During time interval A the cell cavity was filled with a dead volume of poly-T. During time intervals B, D, F, and H, the pump was supplying the cell cavity with fresh poly-T. During time intervals C, E, G, and I, the volume of poly-T in the cell was stationary. During time intervals J, L, N, and P the pump was delivering a 10 nM solution of poly-adenine (poly-A)[12 mer] that was labeled with a 5′-biotin. During time intervals K, M, O and Q, the poly-A solution was static. FIG. 5 shows that gate bias potential changed the most during the first exposure to poly-A, indicating hybridization. The surface becomes saturated and subsequent exposures caused very little change in the bias potential.

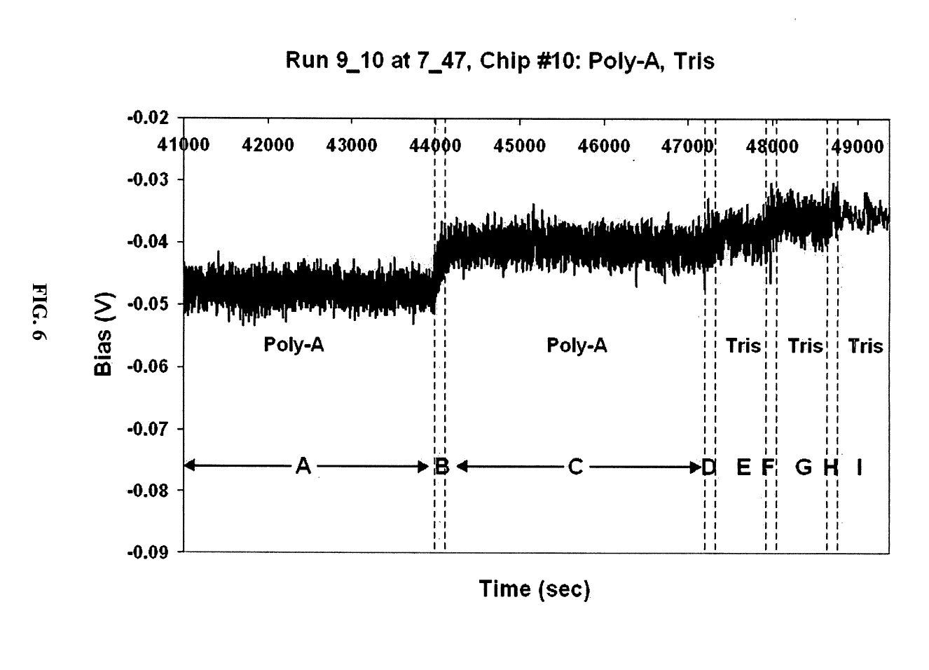

FIG. 6 shows the FET chip post-hybridization. During time intervals A and C, the FET was exposed to static poly-A solution. The pump was running during time intervals B, D, F, and H. During time interval B, the pump delivered poly-A solution. During intervals D, F, and H, the pump delivered tris buffer. During time intervals E, G, and I, the FET was exposed to static tris buffer. FIG. 6 shows that the post-hybridized FET was affected very little by additional poly-A or by pumping or static tris buffer, indicating that a stable, saturated hybridized state was obtained. Hybridization was confirmed by adding 50 μg/ml streptavidin-alkaline phosphatase and subsequent addition of alkaline phosphatase substrate. Alkaline phosphatase binds to the biotin labeled poly A. The generation of formazan (reduced tetrazolium) is monitored—with a positive result yielding a substantial millivolt change—as well as color change. A negative result—no hybridization—does not yield reduction of alkaline phosphatase substrate.

### Example 3

### Determination of Nucleic Acid Hybridization with a Field Effect Transistor

A hydrazine-derivatized FET prepared as described in Example 1 was integrated in the flow cell and exposed to a dead volume of a 10 nM solution of 20-mer of poly-thymidine (poly-T) with a 5′-aldehyde group (SoluLink Biosciences, San Diego, Calif. 92121). The hydrazine-derivatized FET was contacted with the poly-T for 66 hours to produce a hydrazone bond between the surface bound hydrazine and the aldehyde group of the poly-T.

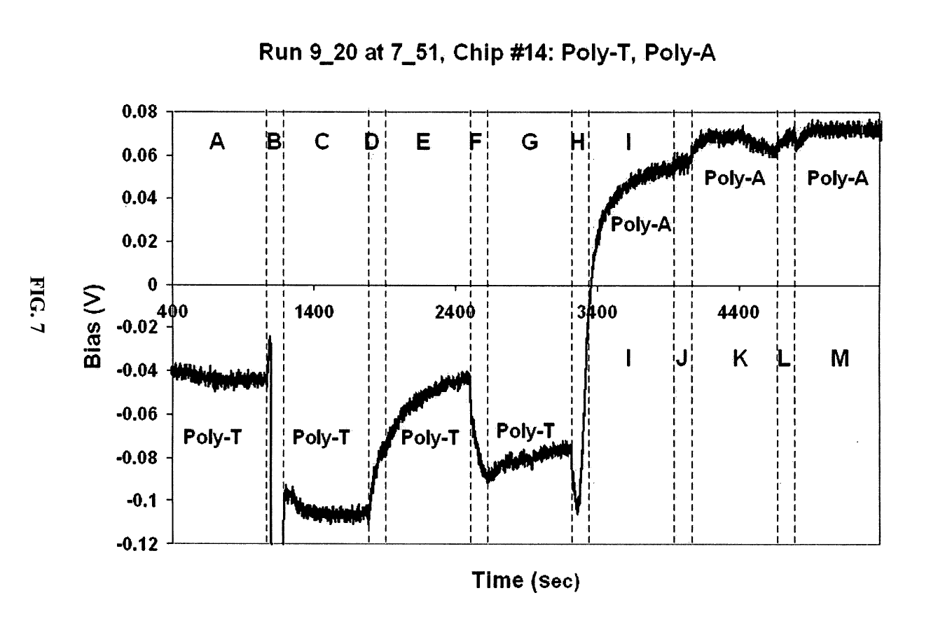

FIG. 7 shows hybridization studies using the poly-T derivatized FET. In time interval A the cell cavity is filled with a dead volume of poly-T. During time intervals B, D, and F the pump supplied the cell cavity with fresh poly-T. During time intervals C, E, and G the cell cavity was filled with a stationary volume of poly-T. This FET showed greater pumping artifacts i.e., greater measured potential spikes due to pumping, but also greater sensitivity than the FET described in Example 2. During time intervals H, J, and L, the pump delivered a 10 nM solution of poly-adenine (poly-A)[12 mer] that was labeled with a 5′-biotin. During time intervals I, K, and M, the poly-A solution was static. As in Example 2, the gate bias potential changed the most during the first exposure to poly-A, indicating hybridization. The surface became saturated and subsequent exposures caused very little change in the bias potential.

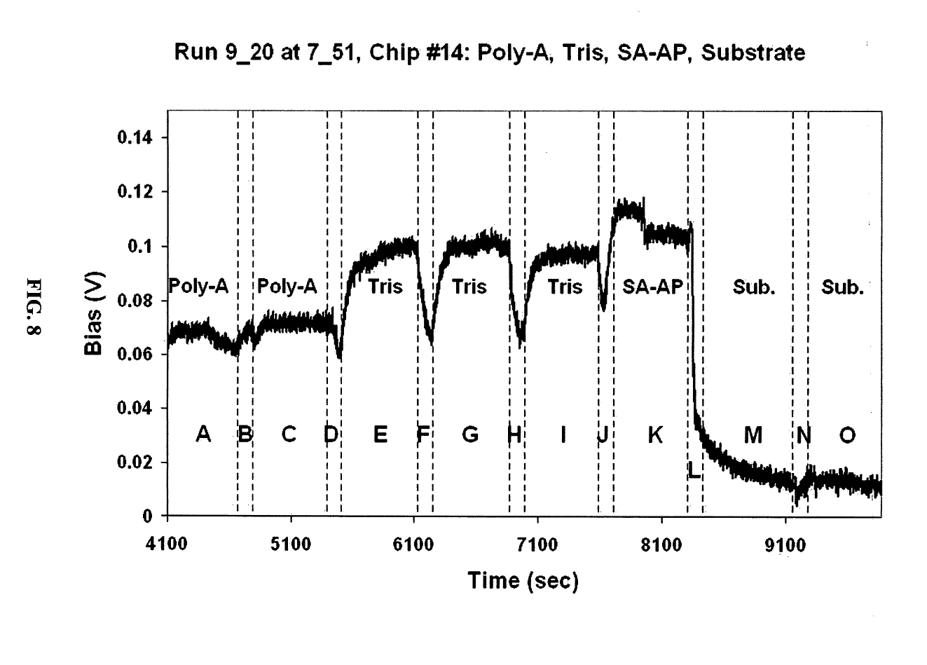

FIG. 8 shows the FET chip post-hybridization. During time intervals A and C, the FET was exposed to static poly-A solution. The pump was running during time intervals B, D, F, and H. During time interval B, the pump was delivering poly-A solution. During intervals D, F, and H, the pump delivered tris buffer. During time intervals E, G, and I, the FET was exposed to static tris buffer. During time interval J, the pump delivered 50 μg/ml streptavidin-alkaline phosphatase conjugate. During time interval K, the FET was exposed to static streptavidin-alkaline alkaline phosphatase. During time intervals L and N the pump delivered alkaline phosphatase substrate. During time intervals M and O, the FET was exposed to a static solution of alkaline phosphatase substrate. Alkaline phosphatase-streptavidin will bind to biotin labeled polyA and react with alkaline phosphatase substrate resulting in measurable millivolt change caused by reduction of substrate—as well as—color change (see e.g., U.S. Pat. No. 5,354,658).

All of the compositions and/or methods and/or processes and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

What is claimed is:

1. A method of attaching a selected molecule to the surface of a substrate, comprising:
selecting a substrate having at least one surface comprising a semiconductor oxide;
contacting the selected surface with a hydrazine compound to produce exposed hydrazine groups bound to the substrate surface;
selecting a molecule capable of forming a stable bond with an exposed hydrazine group; and
contacting the exposed hydrazine groups with the selected molecule.

2. The method of claim 1, wherein the semiconductor oxide is silicon dioxide or germanium dioxide.

3. The method of claim 1, wherein the selected molecule comprises a functional group selected from the group consisting of aldehydes, ketones, carboxylic acids, and urea.

4. The method of claim 1, wherein the selected molecule is selected from the group consisting of oligonucleotides, polypeptides, enzymes, proteins, antibodies, antigens, metabolites, antibiotics, hormones, and drug compounds.

5. The method of claim 1, wherein the selected molecule is an oligonucleotide.

6. The method of claim 1, wherein the hydrazine compound is selected from the group consisting of hydrazine dihydrochloride, hydrazine sulfate, and hydrazine.

7. The method of claim 1, wherein the hydrazine compound is hydrazine dihydrochloride.

8. A method of attaching an oligonucleotide to a silicon dioxide surface, comprising:
contacting the silicon dioxide with hydrazine dichloride to produce exposed hydrazine groups on the surface;
selecting an oligonucleotide having a 5′-aldehyde functional group; and
contacting the hydrazine groups with the selected oligonucleotide.

9. A method of activating a substrate for the immobilization of molecules, the method comprising:
selecting a substrate having at least one surface comprising a semiconductor oxide; and contacting the selected surface with a hydrazine compound to produce exposed hydrazine groups bound to the selected surface.

10. A derivatized semiconductor oxide surface for the immobilization of molecules, comprising exposed hydrazine groups bonded to the semiconductor oxide surface.

11. The derivatized semiconductor oxide surface according to claim 10, wherein the semiconductor oxide surface comprises silicon dioxide or germanium dioxide.

12. A sensor element comprising a semiconductor oxide substrate having at least one surface, and at least one probe molecule chemically bonded to the surface by a hydrazone bond.

13. A sensor for detecting target molecules comprising:
a field effect transistor (FET) having a source implant and a drain implant that are spatially arranged within a semiconductor structure, wherein an active channel separates the source and drain;
a dielectric layer covering the active channel, the dielectric layer having a bottom surface in contact with the active channel and a top surface in contact with a sample solution, wherein the top surface is modified with molecular probes immobilized to the top surface via hydrazone bonds, and wherein the immobilized molecular probes are available to bind target molecules if present in the sample solution; and
a reference electrode in contact with the sample solution, wherein said active channel is biased with respect to the reference electrode.
